(19) United States
(12) Patent Application Publication
(10) Pub. No.: US 2023/0195866 A1
(43) Pub. Date: Jun. 22, 2023

(54) WEARABLE COMMUNICATION DEVICE FOR USE IN CARE SETTINGS

(71) Applicant: Hill-Rom Services, Inc., Batesville, IN (US)

(72) Inventors: Darren S. Hudgins, Cary, NC (US); Amilcar Ubiera, Cary, NC (US); Allen D. Beam, Apex, NC (US); Theophile R. Lerebours, Cary, NC (US); Mark F. Hettig, Denver, CO (US); Catherine J. Harb, Jacksonville, FL (US); Frederick Collin Davidson, Apex, NC (US); Angela E. Kauffman, Sarasota, FL (US); Ryan J. Hoffman, Sarasota, FL (US); Joel Centelles Martin, Barcelona (ES)

(73) Assignee: Hill-Rom Services, Inc., Batesville, IN (US)

(21) Appl. No.: 18/082,037

(22) Filed: Dec. 15, 2022

Related U.S. Application Data
(60) Provisional application No. 63/290,423, filed on Dec. 16, 2021.

(51) Int. Cl. G06F 21/32; G06F 21/44; G16H 40/67; G16H 40/20; G06F 3/16; G06F 3/01; G06F 1/16; H04W 4/02; H04W 4/80

(52) U.S. Cl. CPC G06F 21/32; G06F 1/163; G06F 3/017; G06F 3/167; G06F 21/44; G16H 40/20; G16H 40/67; H04W 4/023; H04W 4/80; G06F 2221/2141

(57) ABSTRACT

A communication device for use in a care facility includes a housing configured to be worn on a caregiver, a display disposed on the housing, a microphone configured to detect sound signals, and a speaker configured to convert an electromagnetic wave input into a sound wave output. The communication device further includes a controller configured to control or receive input from the display, the microphone, and the speaker.

### CROSS-REFERENCE TO RELATED APPLICATION

This application claims priority to and the benefit under 35 U.S.C. § 119(e) of U.S. Provisional Application No. 63/290,423, filed on Dec. 16, 2021, entitled “WEARABLE COMMUNICATION DEVICE FOR USE IN CARE SETTINGS,” the disclosure of which is hereby incorporated herein by reference in its entirety.

### FIELD OF THE DISCLOSURE

The present disclosure generally relates to a wearable communication device and, more particularly, to a hands-free, voice enabled wearable communication device for use in care settings.

### SUMMARY OF THE DISCLOSURE

According to one aspect of the present disclosure, a communication device in the form of a wearable communication device is provided. The wearable communication device includes a housing that is configured to be worn on a caregiver, a display that is disposed on the housing, a microphone that is configured to detect sound signals, a speaker that is configured to convert an electromagnetic wave input into a sound wave output, and a controller. The controller is configured to control or receive input from the display, the microphone, the speaker, and a voice command button. The controller is configured to authenticate the caregiver based on the detected sound signals as an authorized user having a caregiver identification unique to the caregiver.

According to one aspect of the present disclosure, a healthcare communication system is provided. The healthcare communication system includes a plurality of wearable communication devices. Each wearable communication device includes a housing configured to be worn by a caregiver, a display disposed on the housing, a microphone configured to detect sound signals, a speaker configured to convert an electromagnetic wave input into a sound wave output, a beacon configured to emit locating signals, and a controller configured to control or receive signals from the display, the microphone, the speaker, and the beacon. A real-time locating system is in communication with the plurality of wearable communication devices. The controllers of each communication device are communicatively coupled with one another to establish a communication interface between each communication device and, based upon a first location of a first wearable communication device, a voice message is sent to a second communication device, the second communication device including a second location, the second location within a predetermined proximity of the first location.

According to another aspect of the present disclosure, a method of communicating between communication devices over a healthcare communication system is provided. The method includes receiving a voice command from an origin communication device, authorizing the voice command based at least in part on a distinct noise characteristic, identifying an action in response to the voice command, identifying a compatible communication device within a predetermined proximity, and communicating the action with the compatible communication device.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a front view of a wearable communication device, according to the present disclosure;

FIG. 2A is a back view of the wearable communication device of FIG. 1, according to the present disclosure;

FIG. 2B is a side view of the wearable communication device of FIG. 1, according to the present disclosure;

FIG. 3A is a top perspective, exploded view of a wearable communication device having a battery pack, according to the present disclosure;

FIG. 3B is a top perspective, exploded view of the wearable communication device having a battery pack of FIG. 3A, according to the present disclosure;

FIG. 3C is a top perspective view of the wearable communication device having a battery pack of FIG. 3A, according to the present disclosure;

FIG. 4A is a top perspective view of an interior of a wearable communication device, according to the present disclosure;

FIG. 4B is a bottom perspective view of an interior of the wearable communication device of FIG. 4A, according to the present disclosure;

FIG. 5A is a top perspective view of an interior of a wearable communication device, according to the present disclosure;

FIG. 5B is a bottom perspective view of an interior of the wearable communication device of FIG. 5A, according to the present disclosure;

FIG. 6A is a front view of a wearable communication device, according to the present disclosure;

FIG. 6B is a side cross-sectional view of the wearable communication device of FIG. 6A along line VI-VI, according to the present disclosure;

FIG. 7A is a front view of a wearable communication device, according to the present disclosure;

FIG. 7B is a side cross-sectional view of the wearable communication device of FIG. 7A along line VII-VII, according to the present disclosure;

FIG. 8 is a side cross-sectional view of the wearable communication device of FIG. 7A along line VII-VII, according to the present disclosure;

FIG. 9A is a front view of a printed circuit board, according to the present disclosure;

FIG. 9B is a side cross-sectional view of the printed circuit board of FIG. 9A, according to the present disclosure;

FIG. 9C is a back view of the printed circuit board of FIG. 9A, according to the present disclosure;

FIG. 10 is a block diagram of a communication system for the wearable communication device, according to the present disclosure;

FIG. 11 is a block diagram of a communication system for the wearable communication device, according to the present disclosure;

FIG. 12 is a schematic diagram of a healthcare communication system, according to the present disclosure;

FIG. 13 is a schematic diagram of a healthcare room including a caregiver and the wearable communication device, according to the present disclosure;

FIG. 14 is a flow diagram of a method of operating a wearable communication device, according to the present disclosure;

FIG. 15 is a flow diagram of a method of communicating between communication devices over the healthcare communication system, according to the present disclosure;

FIG. 16 is a flow diagram of a method of operating a wearable communication device, according to the present disclosure; and

FIG. 17 is a flow diagram of a method of operating a healthcare communication system, according to the present disclosure.

### DETAILED DESCRIPTION

The present illustrated embodiments reside primarily in combinations of method steps and apparatus components related to a wearable communication device, according to the present disclosure. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

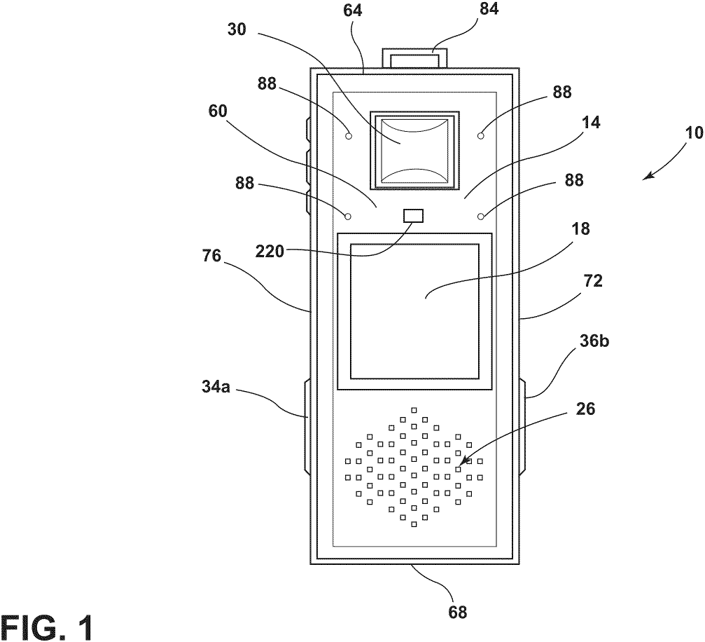

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to a surface closest to an intended viewer, and the term “rear” shall refer to a surface furthest from the intended viewer. However, it is to be understood that the disclosure may assume various alternative orientations, except where expressly specified to the contrary. It is also to be understood that the specific structures and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a ...” does not, without more constraints, preclude the existence of additional identical elements in the process, method, article, or apparatus that comprises the element.

Referring to FIGS. 1-10, reference numeral 10 generally designates a communication device, which may be in the form of a wearable communication device. The communication device 10 includes a housing 14 configured to be worn on a caregiver, a display 18 disposed on the housing 14, a microphone 22 configured to detect sound signals, and a speaker 26 configured to convert an electromagnetic wave input into a sound wave output. The communication device 10 further includes a voice command button 30, at least one help button 34 and a controller 40. The controller 40 is configured to control or receive input from the display 18, the microphone 22, the speaker 26, the voice command button 30, and the at least one help button 34. The controller includes a processor 44 and a memory 48. The controller 40 is configured to authenticate the caregiver based on the electromagnetic wave input to the speaker 26 as an authorized user having a caregiver identification unique to the caregiver.

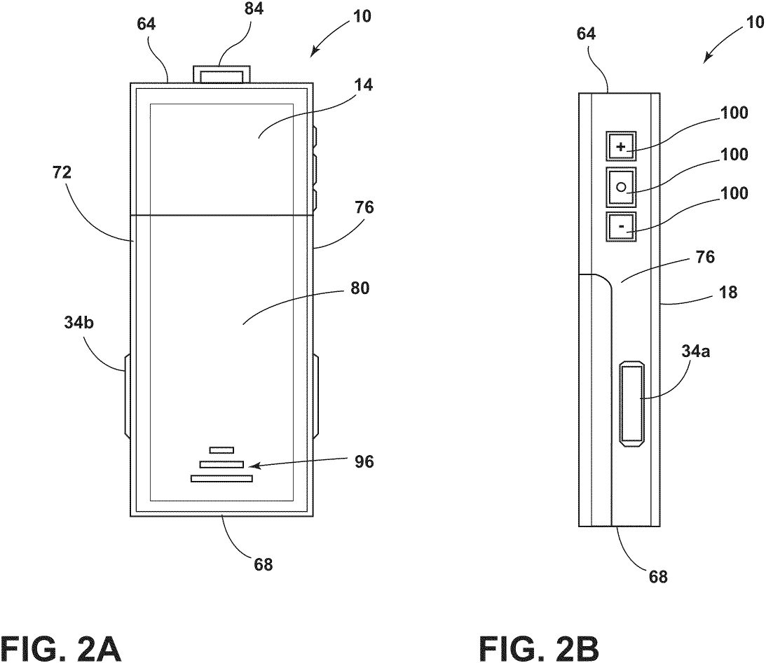

Referring to FIGS. 1-2B, the communication device 10 includes a housing 14 configured to be worn on, or by, a caregiver (e.g., on a portion of the caregiver’s body). As illustrated, the housing defines a front surface 60, an upper surface 64, a lower surface 68, a first side surface 72, a second side surface 76, and a back surface 80. The housing 14 may include an attachment feature 84, which facilitates the use of the communication device as being wearable. In some aspects, the attachment feature 84 includes a circlet, or loop, which may be configured to receive a lanyard, a clip, a band configured to be worn around a body part, such as a wrist, etc. The housing 14 may define a plurality of microphone ports 88 and speaker ports 92.

Further, the housing 14 includes the display 18 configured to display messages, notifications, alerts, and the like. The display 18 may be coupled to and/or integrally formed with the front surface 60 of the communication device 10. This configuration may be advantageous for allowing the caregiver, or user, to grasp the first and/or second side surfaces 72, 76 of the communication device 10 without interfering with the display 18. Further, the caregiver may grasp the communication device 10 and the display 18 may remain visible to the user. In various examples, the display 18 may be configured as a user-interface, such as a touch screen. An anti-slip feature 96 may be provided to facilitate a user’s grip on the communication device 10 and/or to aid in keeping the communication device 10 facing a correct direction when worn on a user’s body.

Further, the display 18 may present a plurality user options. The plurality of user options may include selectable features relating to call contact information, settings, and/or user preferences in non-limiting examples. The caregiver may select one of the selectable features, which may result in a subsequent and different view, or screen, being displayed in response to a user input. In this way, the subsequent screen may be a second level screen relative to the previous screen (e.g., displayed after one user input). The layers of the display 18 may be advantageous for preventing inadvertent activation of a function of the communication device 10. In this way, a plurality of second user options may be displayed in response to a selection of one of a first plurality of first user options, and a third plurality of user options may be displayed in response to selection of one of the plurality of the second user options.

Referring still to FIGS. 1-2B, the housing 14 may also include a variety of selectable features, which may be configured as soft key, buttons, switches, similar tactile features, and/or combinations thereof, etc. As illustrated, the communication device 10 includes a first help button 34a, a second help button 34b, a voice command button 30, and display control buttons 100. The voice command button 30 may be in the form of a tactile button. Optionally, a LED ring may be disposed around the voice command button 30. In some aspects, the display control buttons 100 are in the form of up, down, and select buttons. Further, the display control buttons 100 may also function to control a volume level for output from the speaker 26. When a selection is made by the user, a subsequent display screen may be displayed on the display 18. In this way, selecting the desired selectable feature (e.g., the voice command button 30) may provide access to a corresponding subsequent display screen in order to access various databases related to the care facility, including, but not limited to call contact databases, provider grouping databases, etc.

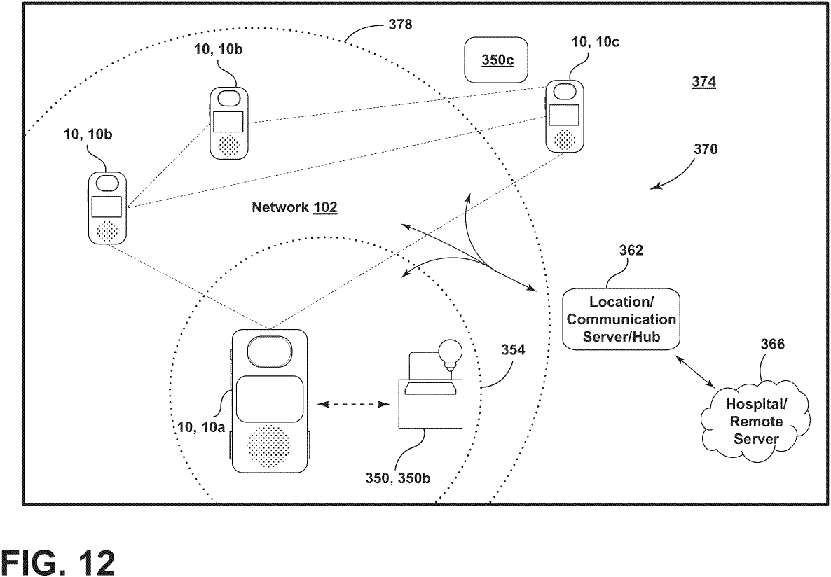

Notifications displayed on the display 18, or emitted through the speaker 26, may include various notifications intended for the caregiver(s). Notifications include messages (e.g. voice, sound or text) from other devices of a network 102 (FIG. 12) and/or other communication devices 10 according to aspects described herein. The messages may include caller, or call information, countdown timer messages (for example, countdown timers from which have reached a minimum threshold), global messages generated for pre-determined groups of staff (for example, all caregivers having a specific certification), automated messages from caregiver monitoring systems, call response messages (for example, information request calls and/or equipment request calls), and direct caregiver messages (for example, messages received from other caregivers).

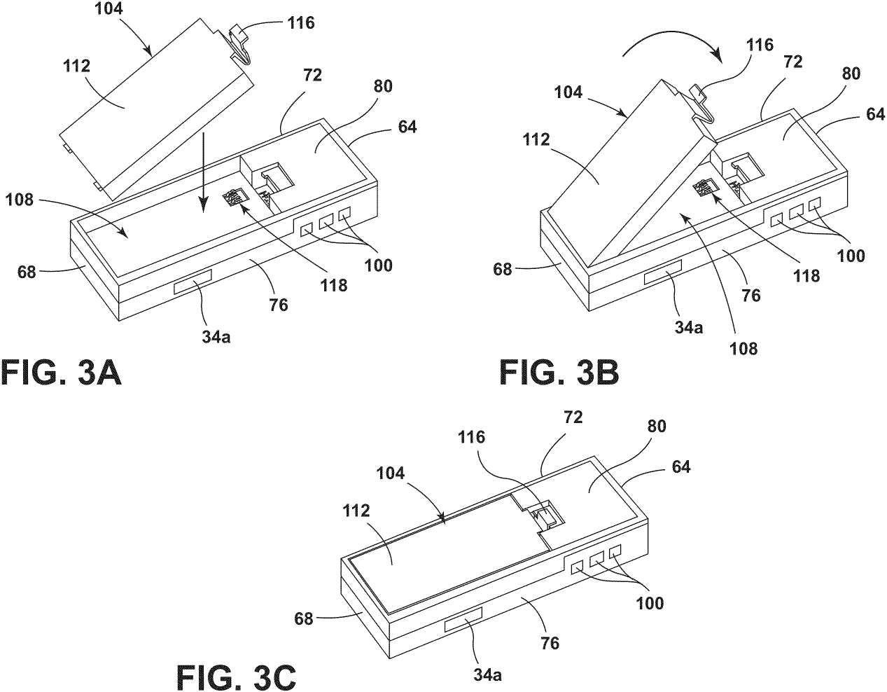

Referring now to FIGS. 3A-3C, the housing 14 may include a battery pack 104 configured to be received within a recess 108 in the housing 14. The battery pack 104 may include a top surface 112 configured to fit flush with the back surface 80 of the housing when fully assembled. In some examples, the battery pack includes a spring clip 116 to retain the battery pack 104 within the recess 108. Alternatively, a fastener (e.g., a screw) is used to lock a battery door in position. A contact spring 118 may be provided in the recess 108, which is configured to contact an electrical connection to a battery disposed within the battery pack 104.

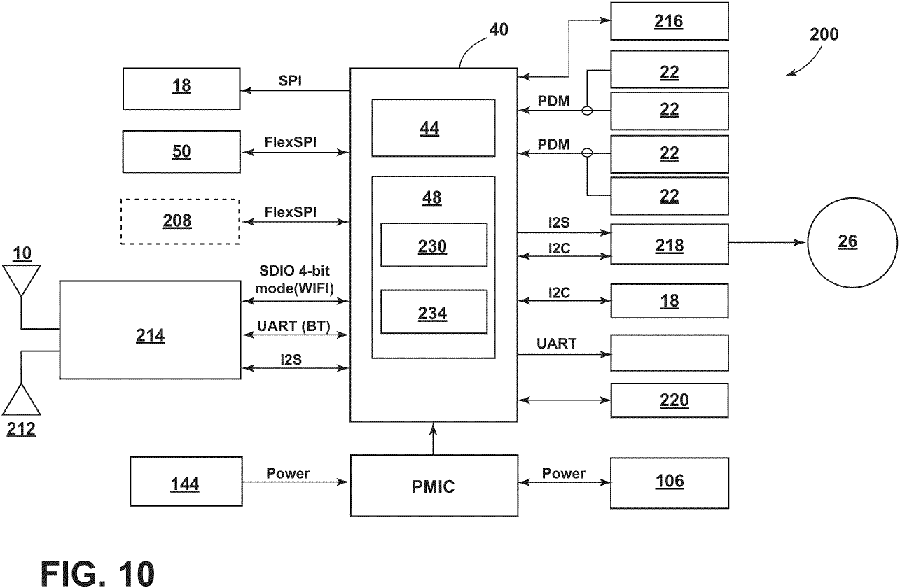

While illustrated as a battery pack 104, a battery 106 (shown schematically in FIG. 10) may be in the form of any suitable battery configured to provide power to the various electronic components of the communication device 10. The battery 106 may be re-chargeable and/or replaceable. In some examples, the battery 106 includes charging contacts or inductive contacts for re-charging the battery. The battery may be sized to prevent interference with a battery enclosure (e.g., the housing 14) after swelling, which may include a height in a range of approximately 18 mm to 18.5 mm. Optionally, a thermistor is disposed within the battery pack 104 to detect battery’s temperature. A PMIC may be responsible for over-temperature protection.

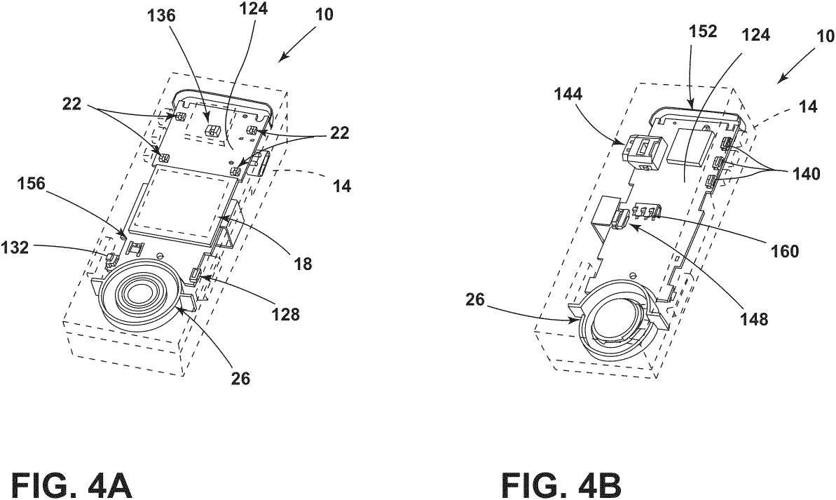

FIG. 4A is a top perspective view and FIG. 4B is a bottom perspective view of an interior 120 of the wearable communication device 10, illustrating the housing 14 in phantom. As illustrated, a speaker 26 and at least one microphone 22 are provided to enable communication between caregivers and multiple communication devices 10. A printed circuit board (PCB) 124 is provided to position, support and electrically connect various electronic components, such as the microphones 22, a first help button switch 128, a second help button switch 132, a voice command button switch 136, and display control button switches 140. The PCB 124 may further support a communication port 144 (e.g., a USB type-C), the display 18, a display connector 148, an antenna 152, a speaker connector 156, a battery connector 160, and the like.

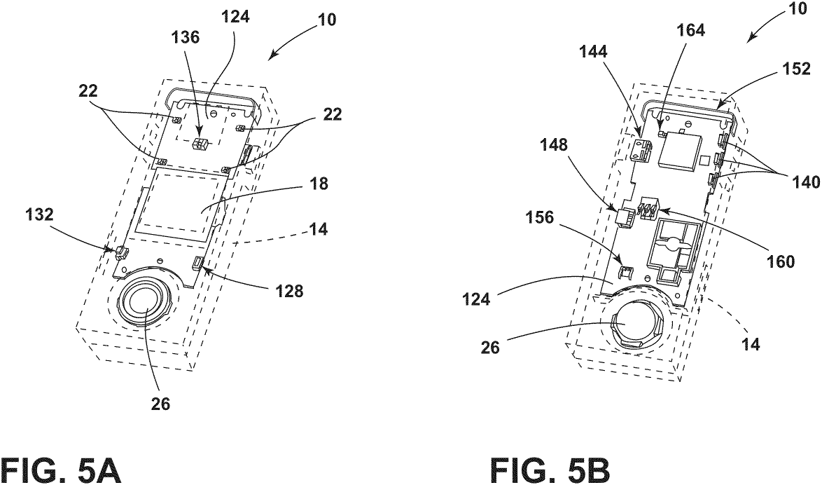

FIG. 5A is a top perspective view and FIG. 5B is a bottom perspective view of the interior 120 of another exemplary wearable communication device 10, illustrating the housing 14 in phantom. As illustrated, the speaker 26 and the at least one microphone 22 are provided to enable communication between caregivers and multiple communication devices 10. The PCB 124 is provided to position, support, and electrically connect the various electronic components, such as the microphones 22, the first help button switch 128, the second help button switch 132, the voice command button switch 136, and display control button switches 140. The PCB 124 may further support the communication port 144 (e.g., a micro-USB), the display 18, the display connector 148, the antenna 152, the speaker connector 156, the battery connector 160, a coaxial cable connector 164, and the like. In some aspects, the coaxial cable connector 164 couples with the antenna 152.

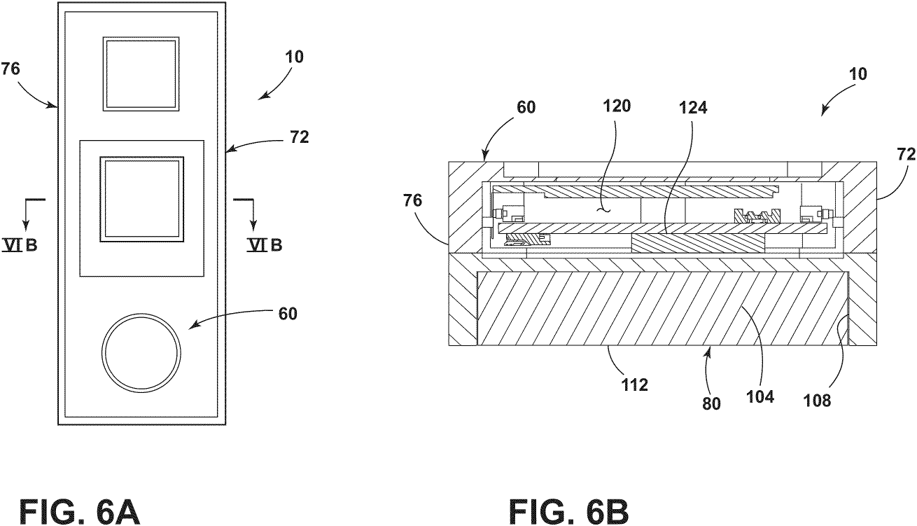

FIG. 6A is a front view of yet another exemplary communication device 10, and FIG. 6B is a side cross-sectional view of the communication device 10 of FIG. 6A along line VI-VI. As illustrated, the housing defines the front surface 60, the first side surface 72, the second side surface 76, and the back surface 80. In FIG. 6B, the battery pack 104 is received within the recess 108 in the housing 14. In this way, the top surface 112 of the battery pack 104 fits flush with the back surface 80 of the housing 14. As may be seen in FIG. 6B, the PCB 124, which is coupled with the various electronic components such as the speaker 26, the microphones 22, the first help button switch 128, the second help button switch 132, the voice command button switch 136, display control button switches 140, the communication port 144 (e.g., a micro-USB), the display 18, the display connector 148, the antenna 152, the speaker connector 156, the battery connector 160, the coaxial cable connector 164 etc. may be disposed within the interior 120 of the housing 14. In this way, the housing 14 protects these components from the exterior environment. In some examples, the housing 14 is generally waterproof or water-resistant.

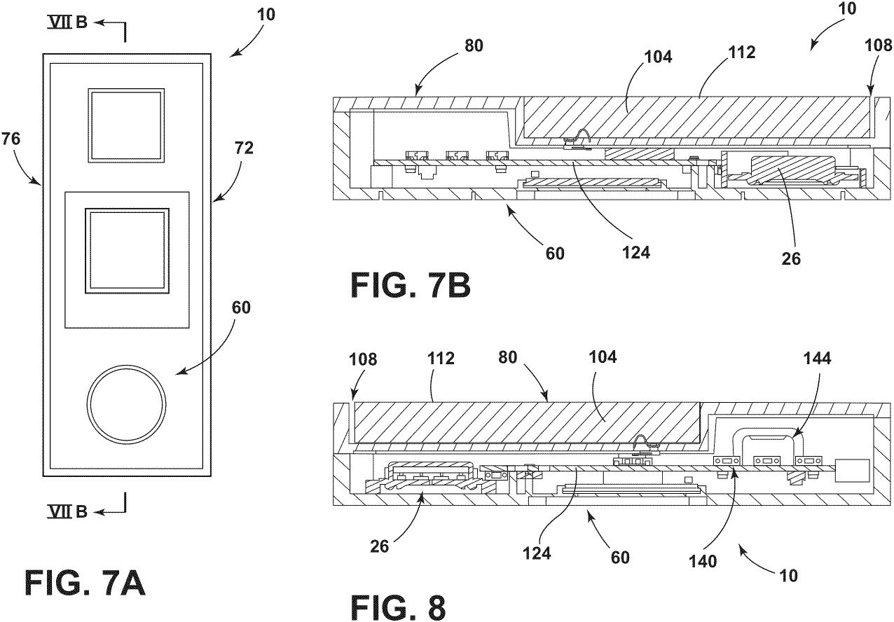

FIG. 7A is a front view of the communication device 10 of FIG. 6A, and FIG. 7B is a side cross-sectional view of the communication device of FIG. 7A along line VII-VII. FIG. 8 also illustrates a side cross-sectional view of the communication device of FIG. 7A along line VII-VII, though mirrored from FIG. 7B. As previously discussed, the housing 14 defines the front surface 60, the first side surface 72, the second side surface 76, and the back surface 80. In FIGS. 7B and 8, the battery pack 104 is received within the recess 108 in the housing 14. In this way, the top surface 112 of the battery pack 104 fits flush with the back surface 80 of the housing 14. As may be seen in FIGS. 7B- 9C, the PCB 124 and the various electronic components, such as the speaker 26, the microphones 22, the first help button switch 128, the second help button switch 132, the voice command button switch 136, display control button switches 140, the communication port 144, the display 18, the display connector 148, the antenna 152, the speaker connector 156, the battery connector 160, the coaxial cable connector 164 etc., may be disposed within the interior 120 of the housing 14. In some implementations, the housing 14 includes a front plate 170 and a back plate 174, wherein the back plate 174 defines the recess 108 configured to receive the battery pack 104. The front plate 170 and the back plate 174 may be joined at a seam 178.

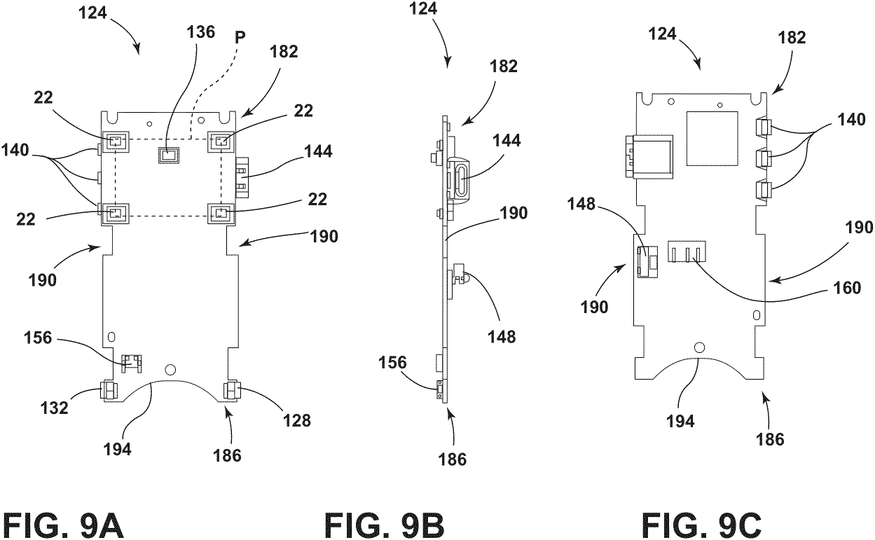

FIG. 9A is a front view of the PCB 124, FIG. 9B is a side cross-sectional view of the PCB 124, and FIG. 9C is a back view of the PCB 124. In some examples, the PCB 124 includes a generally rectangular shape corresponding with the shape of the housing 14. The PCB 124 may define a first end 182, a second end 186, and opposing sides 190. As illustrated, the second end 186 defines a hemispherical recess 194 (e.g., a cutout) shaped to receive the speaker 26. In this way, the hemispherical recess 194 may function to retain the speaker 26 in position and to reduce space requirements within the interior 120 necessary to enclose the speaker 26. In specific implementations, there may be four microphones 22 arranged proximate the first end 182, (e.g., the end opposing the speaker 26) in a quadrant configuration (for example, microphones 22 may be positioned equidistant in each quadrant, I-IV). Stated another way, the microphones 22 may be positioned in an array, which can include a phantom perimeter, P, defining a rectangular, trapezoid, or rhombus placement with two microphones 22 at “upper corners” of the PCB 124 and two microphones 22 on outer edges (e.g., opposing sides 190) closer to the middle of the housing 14. In this way, the microphones 22 can each be positioned to define a corner of the rectangular, trapezoid, or rhombus, etc., shapes. In some aspects, the microphones 22 are at least approximately 20 mm or more away from the speaker 26.

It is contemplated that in some examples of the communication device 10, a horizontal linear distance between two microphones 22 at outer corners of an edge of the PCB 124 is different from a horizontal linear distance between two microphones 22 on opposing sides 190 and proximal to a middle of the housing 14 (e.g., the distances are not equal). Likewise, in some implementations, a vertical linear distance between two microphones on the same side of the housing 14 is different from a vertical linear distance between two microphones on an opposing side of the housing 14. In this way, a phantom perimeter of the microphones 22 can define a trapezoidal shape.

Referring now to FIG. 10, a block diagram of a communication system 200 for the communication device 10 is illustrated. The controller 40 may include communication circuitry to allow the controller 40 of the communication device 10 to communicate with one or more of the communication devices 10 or another remote device 212. One, some, or all of the communication devices 10 within the care facility may include similar controllers 40, processors 44, memory 48, and other control circuitry. Instructions or routines may be stored in the memory 48 and executable by the processor44. In some examples, the communication device 10 includes more than one memory 48. The controller 40 is generally configured for gathering inputs from the various electronic components, processing the inputs, and generating an output response to the input.

The processor 44 may include any type of processor capable of performing the functions described herein. The processor 44 may be embodied as a dual-core processor, a multi-core or multi-threaded processor, digital signal processor, microcontroller, or other processor or processing/controlling circuit with multiple processor cores or other independent processing units. The memory 48 may include any type of volatile or non-volatile memory (e.g., RAM, ROM, PSRAM) or data storage devices (e.g., hard disk drives, solid state drives, etc.) capable of performing the functions described herein. In operation, the memory 48 may store various data and software used during operation of the communication device 10 such as operating systems, applications, programs, libraries, databases, and drivers. The memory 48 includes a plurality of instructions that, when read by the processor 44, cause the processor 44 to perform the functions described herein.

In various implementations, the controllers 40 of each communication device 10 or remote device 212 are communicatively coupled with one another to establish a communication interface 214 therebetween. Therefore, the communication device 10 controller 40 can be configured to communicate with remote servers (e.g., cloud servers, Internet-connected databases, computers, mobile phones, etc.) via the communication interface 214. Specifically, other remote servers include, for example, nurse call computers, electronic medical records (EMR) computers, admission/discharge/transfer (ADT) computers, and the like. The communication interface 214 can include the network 102, which may be one or more various communication technologies and associated protocols. Exemplary networks include wireless communication networks, such as, for example, a Bluetooth® transceiver, a ZigBee® transceiver, a Wi-Fi transceiver, an IrDA transceiver, an RFID transceiver, etc. Additionally, the exemplary networks can include 3G, 4G, 5G, local area networks (LAN) or wide area networks (WAN), including the Internet and other data communication services. Each of the controllers 40 may include circuitry configured for bidirectional wireless communication. Moreover, it is contemplated that the controllers 40 can communicate by any suitable technology for exchanging data. In a non-limiting example, the controllers 40 of each communication device 10 may communicate over the communication interface 214 using infrared (IR) wireless technology. In another non-limiting example, the controllers 40 may communicate over the communication interface 214 via radio frequency (RF) signals. In the IR wireless technology and the RF signal technology, each of the controllers 40 may include a single transceiver, or, alternatively, separate transmitters and receivers.

The speaker 26 of the communication device 10 is configured to convert electromagnetic wave input from the processor 44 into output, such as a sound wave (e.g., audio). In specific implementations, the speaker 26 includes a frequency range from approximately 500 Hz to approximately 3.75 kHz. A peak speaker volume may include 85 dB SPL at 10 cm. An amplifier 218 may be coupled with the controller 40 and the speaker 26 to amplify speaker output. In some examples, the communication device 10 further includes a camera 220. The camera 220 may be communicatively coupled to the controller 40, such that the controller 40 controls operation of the camera 220. Operation of the camera 220 may include turning the camera 220 on or off (e.g., activating and deactivating) and recording (e.g., storing in memory 48) video data received by the camera 220.

In some implementations, the communication device 10 may include an inertial measurement unit 216 (e.g., accelerometer and/or gyroscope, magnetometer etc.). The inertial measurement unit 216 may be configured to detect an acceleration and direction of motion associated with a wearer (e.g., caregiver). Therefore, the processor 44 of the controller 40 may be configured to detect abrupt movements of the communication device 10, which may correspond to a running, flailing, or falling condition of the user. In addition to tracking and detecting abrupt movements, the inertial measurement unit may additionally detect one or more gestures of the user. For example, the gestures may include intentional waving motions, swiping movements, shaking, circular (e.g., clockwise, counterclockwise), rising, falling, or various movements of the communication device 10 in connection with the user. Moreover, the processor 44 of the communication device 10 can analyze and interpret abrupt movements and gestures as a command, or notification.

The microphones 22 are configured to detect sound signals (e.g., voice commands from a user) and send an output signal to the processor 44. In some examples, an effective range of the microphones 22 may include at least 10 meters. In this way, the communication device 10 is configured for far-field sound, or speech, detection. The controller 40 controls operation of the microphones 22. Operation of the microphones 22 may include turning the microphones 22 on or off (e.g., activating and deactivating) and recording (e.g., storing in memory 48) audio data received by the microphones 22. Due to a spatial arrangement of the array of microphones 22, sound can arrive at one or more microphones 22 priorto other microphones 22. In the same way, sound arriving at one or more microphones 22 can include audibly distinct noise characteristics from sound arriving at other microphones 22, which may be based at least partially on an orientation of the communication device 10 relative to a speaker. The microphones 22 provide the characteristic data to the processor 44 as inputs, which can be utilized in downstream decisions of an algorithm to minimize noise and maximize sound intelligibility. For example, the microphone(s) 22 can output one or more time stamps indicative of an arrival time of a sound wave. In another example, the microphones 22 can provide the audibly distinct noise characteristics as inputs to the processor 44.

In some aspects, the communication device 10 processor 44 includes full-duplex voice processing software for digital signal processing (DSP) of the sounds detected by the microphones 22. The processor 44 can determine a noise floor, or the level of background noise (e.g., any signal other than the signal(s) being monitored) and remove specific frequencies caused by the background noise to minimize, or neutralize, the background noise. Moreover, the communication device 10, microphones 22, or microphone array, may be tuned to minimize the background noise in care settings, such as echoes and beeping noises originating from devices within the care setting (e.g., medical equipment). The communication device 10 processor 44 can also analyze three-directional information, including determining a direction that audio is originating from, which can be used for downstream decisions. In this way, the communication device 10 can extract sound sources within an operation range of the microphones 22, such as within a patient room or surgical suite. As previously discussed, audio can arrive at one or more microphones 22 prior to other microphones 22 due to a spatial arrangement (e.g., geometry) of the array of microphones 22. The location of the sound sources (e.g., speaking caregivers, operating equipment) may be inferred by using the time of arrival from the sources to microphones 22 in the array and the distances defined by the array. For example, sound may arrive at a microphone 22 located on the first side 72 of the housing 14 prior to arriving at a microphone 22 on an opposing side (e.g., second side 76). Therefore, a timestamp corresponding to a time of arrival to the microphone positioned on the first side 72 includes a time earlier than a timestamp corresponding to a time of arrival to the microphone 22 positioned on the opposing side. Thus, the communication device 10 can infer that the sound source is nearer to the microphone 22 located on the first side 72 of the housing 14. The communication device 10 and its associated processor 44 may be configured to separate various frequency bands of incoming audio for beamforming applications in order to treat each frequency as individual information when analyzing the sound sources surrounding the microphones 22.

Based on the output signals from the microphones 22 to the processor 44, the processor 44 may transmit a signal over the communication interface 214 indicative of a voice command. As will be discussed in greater detail below, the communication device 10 can be configured to process the output signals from the microphones 22 and authenticate, or recognize, the voice of one or more caregivers by encoding phonetic information and/or nonverbal vocalizations (e.g., laughter, cries, screams, grunts). In one example, the processor 44 reviews the distinct noise characteristics to distinguish between and, ultimately, deduce which caregiver is speaking or vocalizing. In another example, the processor 44 reviews the distinct noise characteristics to distinguish between and, ultimately, deduce a type of person (e.g., female, male, infant, toddler), a person’s biosocial profile, and/or a corresponding emotion (e.g., excited, happy, neutral, aggressive, fearful, and pain) the person is communicating. Audibly distinct noise characteristics may include, but are not limited to, tone, pitch, volume, quality (e.g., timbre), and the like. Fundamental frequency (F0) or “voice pitch” provides clues as to a vocalizer’s identity, which may include sex and age. In general, men produce relatively lower-pitched vocalizations than women. Additionally, a person’s voice F0 can be varied to express a plurality of emotions and motivations during speech. Accordingly, a baseline F0 and a change to the baseline F0 can be determination factors used by the processor 44 to recognize a person and their emotions/motivations. Again, the communication interface 214 may be embodied as any communication circuit, device, or collection thereof, capable of enabling wireless communications between the communication device 10 and remote computers, or other communication devices 10, over the network 102. Therefore, an identity of a caregiver, or other vocalizer, may be communicated to other communication devices 10 or remote devices 212 over the network 102.

The communication device 10 can include an active listening mode. In an active listening mode, the microphones 22 remain in an “on” state. While the communication device 10 can remain “on,” and continually listen, the communication device 10 may not continually record audio data. In some examples, the communication device 10 records and transmits audio only after a “wake word” or phrase/command is identified by the processor 44. In other examples, the communication device 10 records and temporarily stores audio in the memory 48 prior to the wake word being identified by the processor 44, which triggers recording. Audio may be buffered or stored in the memory 48 in approximately 2 second to approximately 10 second intervals, where it is temporarily stored and eventually written over. Therefore, when a wake word or phrase/command is detected, the communication device 10 records the following speech or sounds and transmits a recording over the communication interface 214. In some cases, a wake phrase/command is consistent with a voice command recognized by the processor 44 by comparing the incoming command data with data stored within a voice command database.

When a specific sound signal is determined, or recognized, the processor 44 can initiate a corresponding procedure, or action. The processor 44 can compare the received audio signal to data stored in a voice command database to determine or characterize the sound entering the microphones 22. Accordingly, the microphones 22 can listen for voice commands, or speech, from the associated caregiver, sounds that correspond to a particular situation, such as an emergency or help need (e.g., nonverbal vocalizations), or sounds emitted by devices/equipment located in the care facility for identification by the processor 44.

Thus, the communication device 10 may also identify devices/equipment that is in operation within a range of the microphones 22 when the communication device 10 is in the active listening mode. For example, the communication device 10 may identify incoming sounds as those of an infusion pump or a blood oxygen alert system but is not limited to such. Additional examples of devices/equipment located in the care facility include, hospital beds and mattresses, syringe pumps, defibrillators, anesthesia machines, electrocardiogram machines, vital sign monitors, ultrasound machines, ventilators, fetal heart monitoring equipment, deep vein thrombosis equipment, suction apparatuses, oxygen concentrators, intracranial pressure monitors, feeding pumps/tubes, hemedex monitors, electroencephalography machines, etc. The communication device 10 may initiate an action to adjust settings (e.g., a volume of the speaker 26) of the device 10, provide alerts (e.g., sound or text) to one or more communication devices 10 (e.g., to the display 18 or speaker 26) in response to identifying a device that is in operation.

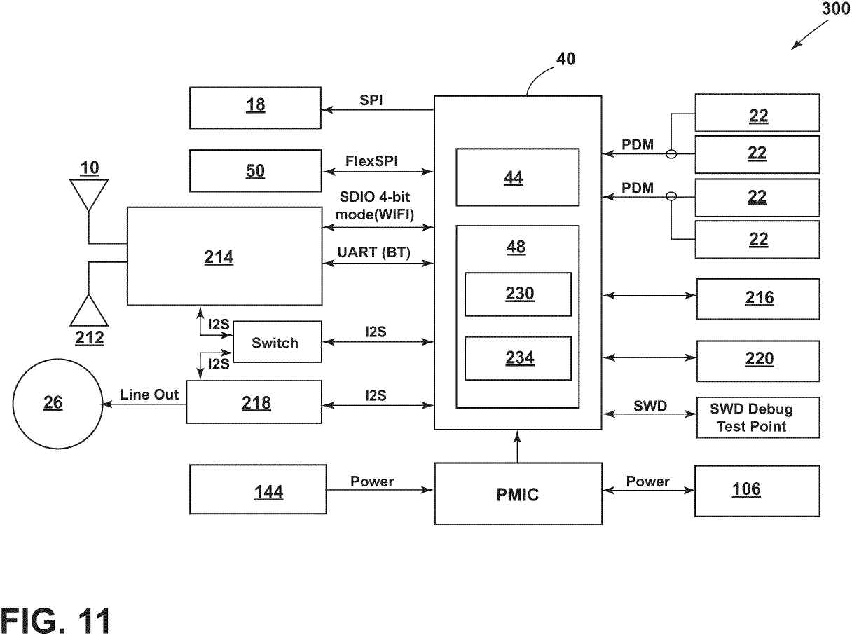

Referring now to FIG. 11, a block diagram of another communication system 300 for the communication device 10 is illustrated. The communication system 300 is similar to the communication system 200. Accordingly, parts identified with like numerals represent like parts, unless specifically stated otherwise. The main difference between the communication system 300 and the communication system 200 is that the communication system 300 includes a switch 222 for I2S that bi-directionally communicatively couples the communication interface 214 (e.g., Wi-Fi) and the amplifier 218. As previously discussed in detail, the controller 40 is generally configured forgathering inputs from the various electronic components, processing the inputs, and generating an output response to the input.

FIG. 12 depicts a diagram of a communication and control system 370 including a plurality of the communication devices 10 communicatively coupled over the network 102. Referring now to FIGS. 10-12, the controller 40 may include a voice recognition module 230. The voice recognition module 230 may be configured to process voice recognition and authentication to recognize, or identify, the voice of one or more caregivers associated with the communication devices 10 and/or a healthcare communication system 370. Further, the controller 40 may include a motion recognition module 234. The motion recognition module 234 can be configured to process received motion data from the inertial measurement unit 216 and analyze it in reference to data stored in a motion recognition database to recognize or characterize the type of movement the communication device 10 is being subjected to.

Caregivers and staff of a facility (e.g., nurses, doctors, technicians, maintenance staff, etc.), upon starting employment with the healthcare facility, can be onboarded, or enrolled, in the healthcare communication system 370 to identify and distinguish between employees. Enrolling employees can include having their voice recorded and stored in an employee identity directory, which is accessible by the communication devices 10.

Further, the employees can be linked to one or more care or service groups during or after enrollment into the healthcare communication system. The care groups associated with each caregiver may be assigned and stored in the directory, which may effectively map the care groups for communication and alert processes. The care groups may be defined based on the specific skills, certifications, security clearance, training, credentials, etc., for each caregiver. Based on the association of each of the caregivers to each of the care groups, communications (e.g., voice commands) that are associated with each of the caregiver’s respective skills may be communicated, or broadcasted, to the communication device 10 that is addressed and assigned to one or more qualified caregivers. In this way, communications over the network 102 may be routed to communication devices 10 assigned to caregivers who are qualified, or skilled, to adequately respond to a particular call or message.

In addition to the association of each caregiver to care groups, the voice data of the caregiver may be linked to the caregiver’s unique identification, which may include information in addition to professional qualifications associated with the care groups, For example, the communication device 10 may identify the voice associated with a caregiver to selectively grant or restrict access to equipment/facilities via the hospital’s access control system, authorize badge access information, computer or hospital network terminal access, voice controlled room control commands (e.g., light control, equipment settings, etc.), and various other information that may be associated with the activities of the caregiver. Additionally, some voice commands, e.g., room control commands, help requests, may be universal to the command databases. In this way, some voice commands may be universal to doctors, nurses, housekeeping, etc.

While some voice commands and communications may be authorized to all caregivers, as noted previously, some voice commands may require recognition of a voice of a caregiver associated with an authorized care group or having the authorization to initiate a request. Based on the identity of the caregiver associated with the voice recorded by the communication device 10, the communication device may authorize a voice command or input into the communication device 10 or access to a device in communication with the network 102. As a result of the association of the voice command to the caregiver, the processor 44 may be instructed to act on a voice command that may be restricted to one or more care groups or caregivers with necessary authorization. In this way, the communication device 10 may prevent false or unauthorized access to alert functions (e.g., sending alerts to improper staff).

In addition to providing authorization, the voice recognition may be implemented to document medical information associated with a patient and the corresponding activities of the caregiver. For example, if a nurse issues a voice command to administer medication, the electronic medical record may be updated to reflect that medication was requested. When the medication is administered, the nurse can utilize the communication device 10 having the voice recognition module 230 to update the electronic medical record with the date, time, and dosage of medication. In some examples, the voice recognition module 230 may be coupled to a real-time locating system server (RTLS) to enable various voice commands. In this way, various voice command databases may be activated based on the whereabouts of caregivers.

For example, a voice command to control a medical device 350 or equipment (e.g., a health monitor, and the like, as previously noted) may only be activated if a nurse or doctor is detected as being within a predetermined distance 354 or proximity (e.g., two meters), of the medical device 350. Accordingly, a voice command to control the medical device 350 (e.g., a drip rate monitor, a hospital bed/mattress) may only be enabled if the nurse or doctor is within the predetermined distance 354 of the medical device 350. The predetermined distance 354 associated with each medical device 350 may vary based on the specific control regime warranted for the type of device. For example, each of the medical devices 350 may be in communication via the network 102 and have corresponding control settings. The control settings may assign the predetermined distance 354 for local or remote voice activation of each of the medical devices 350.

Additionally, the authorization credentials (e.g., care group categories) associated with the user of the communication device 10 (e.g., badge credentials, voice authorization/authentication) may be implemented to unlock or provide access to the user wearing the communication device 10 to access or control the operation of the medical devices or equipment 350. In this configuration, the communication device 10 may capture and detect various voice commands and determine an authorization of the associated caregiver to control the medical device 350. Additionally, the communication device 10 may communicate the credentials of the caregiver to the medical device 350 via a short-range communication 358 (e.g., NFC, smartcard protocol, etc.) to authorize the local use of the medical device 350 via an associated or integral user interface. As understood by the protocols described, the range associated for such short-range communications may be less than 100 cm, 50 cm, 10 cm, or less. Accordingly, the operation of the short-range communication 358 associated with the communication device 10 may be implemented as a complementary communication/authorization function or as a stand-alone access control method. Though not discussed in detail, the voice authentication/identification of the caregiver may serve to cause the communication device 10 to activate the short-range communication 358 required to control the medical device 350. In some examples, the voice recognition module 230 may also be implemented to interpret voice commands and communication control instructions to control various functions within the care facility, such as comfort or operation settings a patient’s room, e.g., entertainment system, lights, window shades, thermostat, and bed controls.

As previously discussed, the programmable operation of each of the communication devices 10 may implemented internally to the controller 40 or distributed among one or more local servers 362 or communication hubs, which may further be in communication with a remote server 366. Accordingly, the operation discussed in reference to the communication device 10 may be provided by the controller 40, servers 362, 366, and/or other connected devices to complete the processes and operating routines described herein. For example, the recognition module 230 may be implemented as one or more specialized processing circuits (e.g., the recognition module 230) or software modules of the communication device 10. Some complex operations (e.g., voice command interpretation) may alternatively be processed via one or more of the servers 362, 366 in communication with the communication devices 10 via the network 102. In this way, the disclosure provides for a flexible solution that may be scaled based on the specific needs of the users and the sophistication of the equipment implemented.

As previously discussed, the operating routines and software associated with the communication device 10 may accessed in the memory 48. In some cases, additional data storage devices 208 may be incorporated in the communication device 10 configured for short-term or long-term storage of data such as, for example, memory devices and circuits, memory cards, hard disk drives, solid-state drives, or other data storage devices. The data storage device 208 may include a plurality of the voice command databases, each having a plurality of voice commands specific to a caregiver type (e.g., role). For example, the commands may include “administer medication,” “CPR,” “change bed,” etc. As previously discussed, separate voice command databases may be accessible for doctors, nurses, or caregivers having specific caregiver identifications. Other voice command databases may be contemplated. For example, a housekeeper voice command database may be provided that is specific to a housekeeper.

The processor 44 of a first communication device 10, in response to an output signal from the microphone 22, may be instructed to relate the output signal to a voice command in the data storage device 208 and transmit a signal to a second communication device 10 or a remote computer indicative of the voice command. At the second communication device 10 or the remote computer, a processor of the second communication device 10 or remote computer 36 relates the output signal to a voice command in the data storage device 208. In this way, the second communication device 10 may then activate a graphic or audible alert indicative of the voice command.

As demonstrated in FIG. 12, the healthcare communication system 370 may include a plurality of the communication devices 10 to connect a variety of caregivers, specifically providing voice communication in situations where use of hands is not practical. Further, the healthcare communication system 370 may operate as a high-accuracy locating system which is able to determine the location of each communication device 10, which may be accurate within approximately 1 meter. Accordingly, the healthcare communication system 370 may be used to track the whereabouts of multiple caregivers and other staff members, or users, wearing a specified communication device 10 in the care facility.

The healthcare communication system 370 may include a plurality of the communication devices 10. However, each caregiver may not be assigned a unique communication device 10 for individual use. It is advantageous to the system to enable a caregiver, or other employee, to utilize any one of the communication devices 10 available for use at a time of need (e.g., during their shift). In some aspects, the communication device 10 can locate a caregiver’s badge using a short-range protocol (e.g., Bluetooth, ultrawideband, near-field communication, etc.) and pair with the located badge for onboarding, or provisioning purposes. In this way, the communication device 10 can quickly be associated with the caregiver to use during a limited period (e.g., a shift) and unpaired when the limited period of use is complete. Therefore, the healthcare communication system 370 needs only a limited number of communication devices 10 as a variety and number of employees can utilize the same communication devices 10 (e.g., each employee does not need a personal device). In some aspects, the healthcare communication system 370 can compare data relating to the communication devices 10 in use associated with an employee identity with data stored in the hospital’s barrier access control system, or entry system, to determine an employee present in the facility who is not currently holding (e.g., provisioned) a communication device 10.

The healthcare communication system 370 may be communicatively coupled with a nurse call system, a master nurse station or computer, an electronic status board in communication with the master nurse station and/or server, indicator assemblies, such as dome lights adjacent the doorways of the various patient rooms, input/output (I/O) boards coupled to the room stations and dome lights, bathroom call switches, shower call switches, etc. Further the healthcare communication system 370 may include computer devices such as desktop computers, laptop computers, computers on wheels, mobile phones, and personal digital assistants.

As previously discussed, the communication devices 10 may be connected to the real-time locating system (RTLS), which may be implemented on the local servers 362 or communication hub. That is, the local server 362 may correspond to a location server including a network of computers or remote readers (e.g., mobile phones, tablets) distributed throughout and forming a sensory network within a location of the healthcare communication system 370. Each of the readers of the healthcare communication system 370 may detect the relative location of the communication devices 10 and, therefore, the associated caregiver identity (e.g., badge identification) and location(s) of medical devices 350 and equipment that may be installed in fixed positions or moveable throughout a facility. The readers of the location server 362 may be distributed among different floors and locations on each of the floors, such that the locations of each of the communication devices 10 and medical devices 350 may be tracked in real-time. In some aspects, the remote device 212 is configured to emit a compatible signal (e.g., UWB) with the communication device 10. Accordingly, the remote device 212 can be leveraged to authenticate or supplement location information determined by the location server 363 of the healthcare communication system 370.

For example, a caregiver may have a remote device 212 that includes programming for a healthcare facility employee application that stores data relating to a caregiver, such as badge identification number, credentials, birthday, name, work schedule, security clearance, cafeteria account, etc. The remote device 212 can utilize its short-range communication capabilities (e.g., UWB, Bluetooth, RFID, NFC, etc.) to communicate data to the healthcare communication system 370 about an employee such as their qualifications to the communication device 10 within a short range. The cooperation (e.g., tethering, pairing) of the communication device 10 and the remote device 212 over short-range communication may be implemented in a range of approximately 10 meters or less, 1 meter or less, 10 cm or less, or 5 cm, or less. The data from the remote device 212 may be communicated to the healthcare communication system 370 to authenticate a location determined by the location server 363 as that of the caregiver associated with a communication device 10. In another example, the data from the remote device 212 may be communicated to the healthcare communication system 370 to identify an employee associated with a remote device 212 having the health care facility employee application who does not have a communication device 10 assigned to them at that time. Therefore, the remote device 212 can provide an additional factor for authentication and tracking of personnel by the healthcare communication system 370. Moreover, it is within aspects of the disclosure for the healthcare communication system 370 to communicate audio or text messages and the like to the remote device 212 (e.g., operating as a walkie talkie).

The system 370 may provide for asset (e.g., medical device 350) and personnel (via the communication device 10) tracking using the location server 362. As a component of this tracking process, the healthcare communication system 370 may identify and track the precise location of the communication devices 10 in real-time. The asset and personnel tracking features can be leveraged to determine a distribution of resources associated with the healthcare facility. As previously described, the communication devices 10 can be associated with caregivers having particular credentials or qualifications, which are considered resources. The healthcare communication system 370 can analyze a distribution of these resources throughout the healthcare facility by comparing the information provided by the communication devices 10 pertaining to the users’ particular credentials and qualifications to the precise location of a plurality of the communication devices 10. In some aspects, the healthcare communication system 370 analyzes all the communication devices 10 within the healthcare facility to determine a total of available resources. In other aspects, the healthcare communication system 370 analyzes only the communication devices 10 within a particular region, or ward (e.g., a room, a floor, a cardiothoracic unit, a neonatal intensive care unit, a surgical unit, a long-term care unit, etc.).

A beacon 50 (e.g., RFID, ultra-wideband [UWB] transmitter, etc.) may be integrated within the communication devices 10. The beacons 50 are configured to send signals over the communication interface 214. In this way, other communication devices 10 or the remote device 212 over the communication interface 214 can retrieve locating information from the RTLS for use by the processor 44 of the communication device 10. The healthcare communication system 370 can locate the beacons 50 positioned within a predetermined range (e.g., a particular region or ward) and analyze the credentials associated with the communication devices 10 corresponding to those beacons 50. A map module accessible by the processor 44 can store data regarding the layout of the health care facility, which can include geographical coordinates. For example, the processor 44 can correlate a coordinate of a beacon 50 with a coordinate associated with a position stored within the map module. In this way, the health care communication system 370 can infer, or determine, a distribution of resources relative to the layout of the healthcare facility. A location, such as a room stored in the map module of the healthcare communication system 370 can also be associated with patient needs, which can be based at least in part on patient conditions, or procedures undertaken in the room. In this way, the healthcare communication system 370 can determine the number and type of resources (e.g., staff members having the necessary skill sets, or qualifications) to adequately attend to patients in various regions, locations, or departments in the facility.

The healthcare communication system 370 can use the information regarding the distribution of resources to initiate an action to allocate the resources within the healthcare facility. The healthcare communication system 370 can also use the location data received from the communication device 10 to make a determination on whether or not a particular caregiver (e.g., badge/device holder) is within a predetermined location. A predetermined location may be an appropriate or requested location but is not limited to such examples. The location data can also be used to determine whether the particular caregiver is moving in a direction toward or away from the predetermined location. The healthcare communication system 370 and associated processor(s) 44 may be configured to determine a conflict between a caregiver’s current location and the predetermined location. A conflict may be that the caregiver is not in the predetermined location or is moving away from the predetermined location. For example, a caregiver associated with a neonatal unit is in a break room, but the health care communication system 370 determined that the caregiver is supposed to be positioned in the neonatal unit.

The healthcare communication system 370 can use the location data received from the communication device 10 to track whether the associated caregiver is in an incorrect room/region and provide a corresponding notification/alert. Additionally, the healthcare communication system 370 can use the location data received from the communication device 10 to track whether the associated caregiver completed their rounds by visiting each and every required room. Therefore, the communication device 10 can notify the associated caregiver if a room (or rooms) was missed from their rounds. The communication device 10 can also notify the associated caregiver if their round is complete. The healthcare communication system 370, including the plurality of the communication devices 10, may provide for placing or receiving calls via the communication device 10 from a channel. A channel may be a physical location or logical grouping. A voice call may be placed from one communication device 10 to another communication device 10 assigned to a particular staff member, or role, using voice commands. Likewise, a caregiver may answer an incoming call by way of a voice command input to the communication device 10.

The communication devices 10 may also be used to send group messages associated with a logical grouping (e.g., a location, a caregiver role, care group). The messages can be in the form of text, or optionally, announced as a voice message (i.e., text to voice) at the communication device 10. A source of the messages may be a mobile device (e.g., tablet, smartphone, laptop), a desktop computer, another communication device 10, or any devices or medical equipment in communication with the network 102. In some examples, the caregiver may activate the voice command button 30 and speak a message intended for one or more recipients chosen by the logical grouping. A caregiver may choose the logical grouping using a variety of methods, which may include performing a specific number of clicks configured to call different endpoints, or contacts from a logical grouping.

Still referring to FIG. 12, the RTLS tracking features of the healthcare communication system 370 may be implemented to identify the positions of each of the communication devices 10 and medical devices 350 within a monitored region 374 of a facility. As previously discussed, each of the communication devices 10 may be programmed with information identifying the identity of the user as well as the credentials and/or care groups with which the user/wearer is associated. In this way, the communication device 10 may be in the form of a locating badge. Based on this information, the healthcare communication system 370 may track the credentials or care groups associated with each of the communication devices 10 as well as the medical equipment within a predetermined distance depicted in FIG. 12 as element 378.

The healthcare communication system 370 can manage a distribution of caregivers using the communication devices 10. For example, the healthcare communication system 370 may identify a first location (e.g., a first hospital room) having a group of multiple, or additional, caregivers associated with a same care group (e.g., nurses). The healthcare communication system 370 may also identify a second location (e.g., a second hospital room) having zero, or less than a desired number of, caregivers associated with the same care group. In response to these determinations, the healthcare communication system 370 can communicate with the communication device(s) 10 to send a notification (e.g., audio or text) to request that the caregiver associated with the communication device(s) 10 moves to the second location.

As depicted in FIG. 12, the predetermined distance 378 may correspond to any distance associated with a specific call request or urgency associated with a call transmitted via the communication device 10. For clarity, the distance will be referred to as a call range 378 (e.g., 30 m) that may be programmed to be greater (e.g., 50 m, 80 m, facility wide) or less (e.g., 20 m, 10 m) relative to the position of the communication device 10 from which the call originated (e.g., origin device 10a). The distance or reach of the call range 378 may also be controlled to extend in relation to a floor on which the origin device 10a is located, adjacent floors, or floors that are within the call range 378. In this way, the call range may be assigned for each call, command, request, or code communicated from the origin device 10a to reach personnel within a distance and location that may effectively serve to respond to the request. For clarity, devices 10, 350 may be distinguished as being within range 10b, 350b or outside of range 10c, 350c. Accordingly, the healthcare communication system 370 may be configured to track the positions of each of the device 10, 350 to determine whether they are within the call range 378.

Based on the location of the origin device 10a, the system 370 identify the devices 10b, 350b within range of the call or command. Additionally, because the location of each of the devices 10 is tracked in real-time, the system 370 may identify the devices 10c, 350c that are outside of the call range 378. In operation, the system 370 may receive a call, command, or request via the origin device 10a. The call or command may be identified based on a voice recognition, user input, gesture (as later discussed), impact or acceleration, or interaction with the origin device 10a. Once the call command or request signal is received by the location server 362, the system 370 may identify the call range setting associated with the call or command. The call range 378 may be programmed differently for different types of requests, alert levels, and/or the urgency associate with a request. Once the distance or range associated with the call range 378 is identified, the system 370 may further determine which of the devices 10b, 350b are associated with caregivers or users with the credentials or qualifications necessary to or authorized to answer the call. Accordingly, the location server 362 may determine the devices 10b, 350b are within the call range 378 and also identify which of the devices 10b, 350b within the call range 378 are associated with caregivers with qualifications necessary to respond to the call. Accordingly, the system 370 may communicate a corresponding alert, command, instructions, request, or information to the devices 10b, 350b in the call range 378 and associated with the caregivers with the credentials or in the caregiver group that is assigned to respond to the call.

In some examples, the call or command from the origin device 10a may be configured to control the medical devices 350 or various automated equipment of the facility. For example, in response to a request or command, an alert condition for the facility, a department, or a floor of the facility may be initiated by the system 370. In response to the alert condition or as a result of a specific voice or control command from the communication device 10, the system 370 may activate one or more facility doors to open or close. For example, as a result of a lockdown command from a user of a communication device 10 identified by the location server 362 as being located in a particular department, the system 370 may communicate an instruction to close one or more doors or barriers defining a perimeter of the department. In this way, the system 370 may provide for automated facility controls (e.g., door control, barrier control, alarms, etc.) to be activated in response to the command, request, or voice instruction received by the communication device 10.

As previously described, the communication device 10 may include the inertial measurement unit 216 (e.g., accelerometer and/or gyroscope, magnetometer, etc.). The inertial measurement unit 216 may be configured to detect the acceleration and direction of motion associated with a wearer. In this configuration, the processor 44 of the controller 40 may be configured to detect abrupt movements of the communication device, which may correspond to a running, flailing, or falling condition of the user. In some cases, the communication device 10 may also be implemented as a wearable (e.g., wrist, bracelet, lanyard, clip-on) device connected to the user and configured to detect one or more gestures. Upon detecting the gestures and/or motion data, the communication device 10 may initiate one or more requests, commands, actions, and/or controls that may be communicated to other communication devices 10 or medical devices 350 in communication via the network 102. For example, in response to the detection of an abrupt movement (e.g., a fall), the communication device 10 may initiate a request for assistance to the location of the caregiver identified at the time of the detection of the acceleration associated with the abrupt movement. In response to the call from the origin device 10a, the location server 362 may communicate an audible or text alert to the users of the communication device 10b within the call range 378 to move to the location (e.g., room number, hall, department, etc.) from which the automated call originated.

Again, the inertial measurement unit may also detect one or more gestures of the user. For example, the gestures may include waving motions, swiping movements, shaking, circular (e.g., clockwise, counterclockwise), rising, falling, or various movements of the communication device 10 in connection with the user. In response to the detection of the gesture, which may be intentional, the communication device 10 may be configured to identify a corresponding control instruction or request. A gesture can be more subtle than a caregiver audibly requesting help, which may be advantageous, in some instances. For example, a flailing motion exceeding a predetermined time duration may initiate a help request from an origin device 10. In another example, a caregiver can intentionally grab a communication device 10 and shake the device. The intentional shaking motion can be recognized by the motion recognition module 234 as an emergency and request for assistance. In some aspects, the motion recognition module 234 may be coupled to the RTLS using the location server 362 to provide location information of the origin device 10a and to identify devices (e.g., devices 10b) within the call range 378 that may quickly respond to the request. In addition, the healthcare communication system 370 and associated processor(s) 44 can make a determination that a communication device 10b that acknowledged the request for assistance is moving in a conflicting direction with respect to the location of the origin device 10a. As such, an alert can be communicated to the communication device 10b, and, therefore, the associated caregiver that they are moving in a wrong direction. In some examples, the alert continues until the associated caregiver is moving in a proper direction with respect to the location of the origin device 10a.

Additionally, the gestures may be detected to activate the short-range communication 358 required to control the medical device 350. Similarly, the gestures may be detected by the communication device 10 to control a medical device 350 or, more generally, a computerized device within the predetermined distance 354. In this configuration, the system 370 may identify the location of the communication device 10 associated with the gesture and determine if a corresponding medical device 350 or computerized device is within the predetermined distance 354. In response to the detection of the gesture, the system 370 may communication corresponding control instructions to the medical device or computerized device to initiate gesture control.

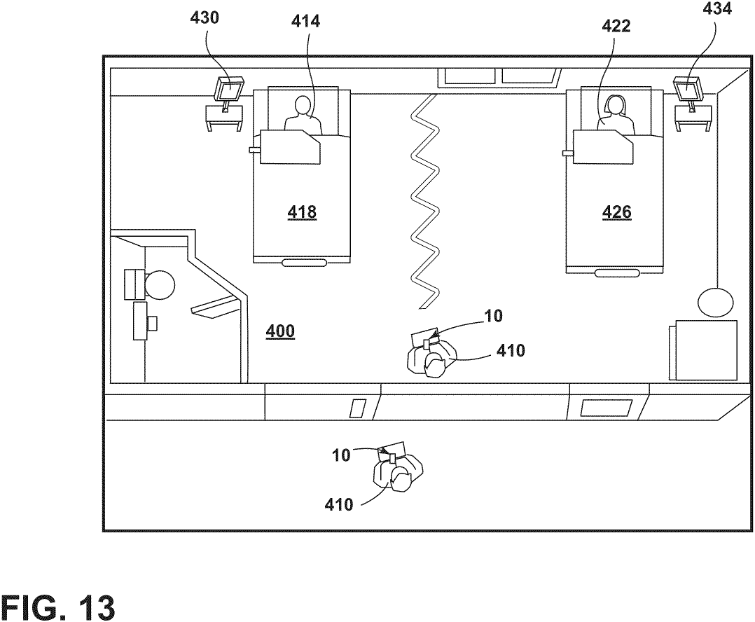

Referring now to FIG. 13, a schematic healthcare room 400 is illustrated. A caregiver 410 holding/wearing the communication device 10 is positioned in the room 400. In addition, the room 400 includes a first patient 414 positioned on a first bed 418 and a second patient 422 positioned on a second bed 426. As illustrated, a first vital sign monitor 430 is associated with the first patient 414 and a second vital sign monitor 434 is associated with the second patient 422. The communication device 10 can be in an active listening mode.

In some aspects, the communication device 10 is configured to distinguish between the first patient 414 and the second patient 422. For example, the first patient 414 is a male, aged 55, who is speaking aggressively, while the second patient 422 is a male, aged 32, who is not speaking, or speaking in a neutral tone. The communication device 10 may recognize that the aggressive speech is originating from the first patient 414 based, at least in part, on the audibly distinct noise characteristics including pitch, which can provide clues as to a vocalizer’s sex and age as previously described in much detail. Therefore, the communication device 10 can provide information to the healthcare communication system 370 regarding the patient’s behavior. In another example, communication device 10 may recognize that the aggressive speech is originating from the first patient 414 based, at least in part, on the direction that the audio is originating from as previously described in much detail with respect to the microphone 22 array.

In other aspects, the communication device 10 is configured to distinguish between the first vital sign monitor 430 and the second vital sign monitor 434. Accordingly, in the event that the second vital sign monitor 434 is producing an emergent alert, which may correspond to an indication that the second patient 422 is experiencing cardiac arrest, a communication device 10 that is within range, regardless of whether the caregiver associated with the device is able to visualize the second patient 422 and/or the second vital sign monitor 434 (e.g., the caregiver 410 is in a hallway), can notify the caregiver that the second patient 422 requires immediate cardiopulmonary resuscitation (e.g., without the caregiver needing to deduce which patient is in need). In this way, a time of arrival to the second patient 422 can be decreased. Optionally, the communication device 10 is also configured to determine a room number from which the emergent alert is originating. The communication device 10 can distinguish between the first vital sign monitor 430 and the second vital sign monitor 434, as well as detect an associated room number at least based in part by identifying incoming sound uniquely associated with the second vital sign monitor 434 and/or by inferring the location of the sound sources as previously described in much detail with respect to the microphone 22 array.

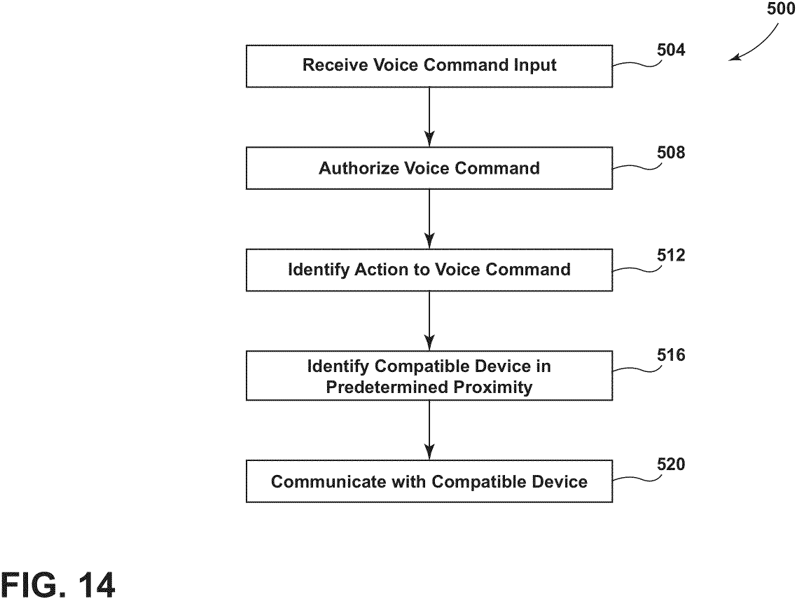

Referring to FIG. 14, a method 500 of operating the communication device 10 is illustrated. The method 500 may begin at step 504 where a first communication device 10 receives a voice command. The microphone 22 sends an output signal to the processor 44 indicative of this command. The first communication device 10 may authorize the voice command at step 508. The communication device 10 may authorize the voice command based upon a variety of parameters using the voice recognition module 230. Next, at step 512, identifying an action in response to the voice command is determined. In some examples, an action is a voice call or a voice message, which may include a request. For example, a request may include a request for administration of CPR. As such, the caregiver may say, “administer CPR.” Then, at step 516, the communication device 10 may use the communication interface 214 to identify at least one compatible communication device 10 within a predetermined proximity or vicinity. In some examples, all compatible communication devices 10 within the predetermined proximity are identified. At step 520, the first communication device 10 communicates with the compatible communication device(s) 10.

Optionally, the method may include a step of sending an acknowledgement to the each of the compatible communication devices 10. The acknowledgement may include an alert that the action has been engaged by one of the each of the plurality of compatible communication devices. In this way, the caregiver assigned to the first communication device 10 and the caregivers assigned to the rest of the compatible communication devices 10 may be informed that a request is being addressed. Further optionally, the action may include location information, such that the compatible communication devices 10 also receive the location information.

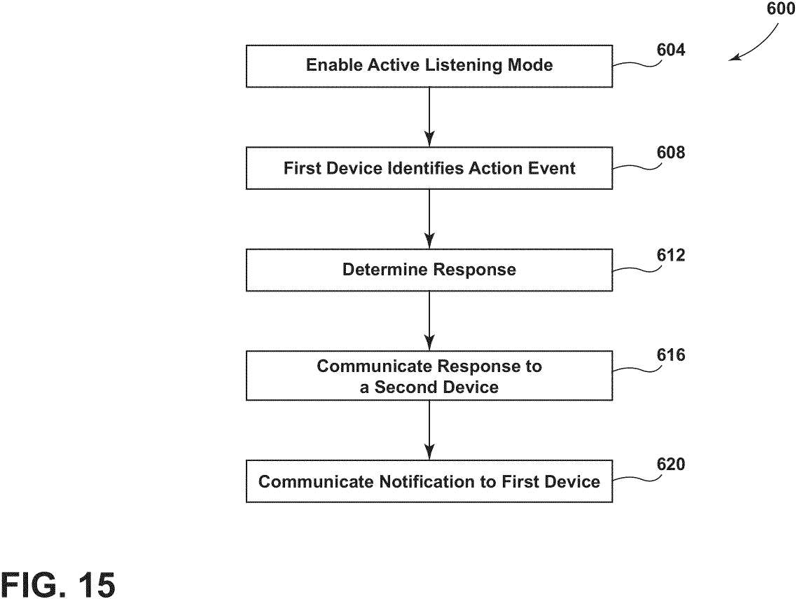

Referring to FIG. 15, a method 600 of communicating between communication devices 10 over the healthcare communication system is illustrated. The method 600 may begin at step 604 where an active listening mode is enabled on a first communication device 10. In this way, the controller 40 controls the microphone(s) 22 to remain on (e.g., listening). The first communication device 10 (e.g., origin device 10a) may identify an action event at step 608. In some examples, the action event is a scenario, or sound, that corresponds to duress (e.g., loud noises, cries, screams, aggressive voice recognition). In this way, active listening can characterize a type of distress, which is typically much louder and higher pitched than natural speech. In another example, the action event is a scenario, or sound, that corresponds to a coded alert. Various alerts, including coded alerts, can be characterized by a tone, volume, frequency, etc., of the alert. Next, at step 612, the first communication device 10 may determine a response to the action event and escalate the response accordingly. For example, a response may be to summon the nearest security personnel. Then, at step 616, the response is communicated to at least a second communication device 10 (e.g., a communication device associated with the nearest security team member). In some examples, a plurality of communication devices 10 within the predetermined proximity are identified and communicated with. In some implementations, the first communication device 10 can utilize the short-range communication 358 and/or the beacon 50 (e.g., UWB) to filter out, or reduce, a number of communication devices 10 located within the predetermined proximity to devices 10 that are the closest to the origin device 10a. Further, the first communication device 10 can utilize the short-range communication 358 to check a specific position identified as within the predetermined proximity 378 using the location server 362 to avoid false positive results. At step 620, the second communication device 10 may communicate a notification to the first communication device 10 regarding an acknowledgement to the response by the second communication device 10. For example, an audio message stating “help is on the way.”

Optionally, the method 600 may include a step of recording audio from the microphones 22 or even visual feed from the camera 220, once an action event is identified at step 608. In some aspects, the response communicated to the at least the second communication device 10 includes the audio or visual in order for the caregiver assigned to the second communication device 10 to “witness” the event. Witnessing the event may include listening to and/or viewing the live audio/visual from the first communication device 10.

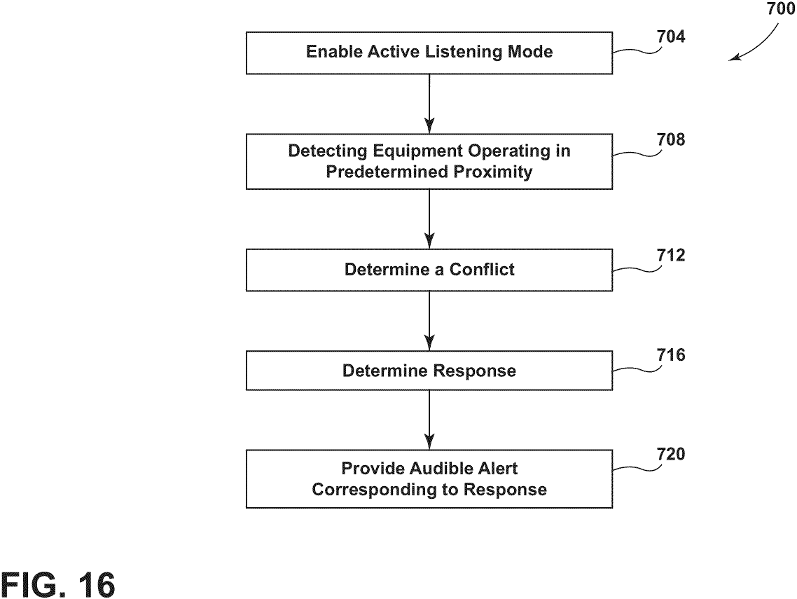

Referring now to FIG. 16, a method 700 of operating the communication device 10 illustrated. The method 700 may begin at step 704 where an active listening mode is enabled on a first communication device 10. In this way, the controller 40 controls the microphone(s) 22 to remain on (e.g., listening). The first communication device 10 may detect at least one equipment operating in a predetermined proximity at step 708. The detection may include an analysis of noises, or sounds, which may be unique to the equipment, such that the first communication device 10 identifies the equipment. Next, at step 712, the first communication device 10 may determine a conflict. For example, the conflict may be in the form of two types of equipment operating at the same time that should not be. For example, the at least one equipment may include a blood oxygen warning alert and the conflict may include a delivery of medicine. Then, at step 716 a response to the conflict may be determined (e.g., a warning to not deliver medicine because the blood oxygen warning alert is on). An audible alert corresponding to the response may be provided at step 720.

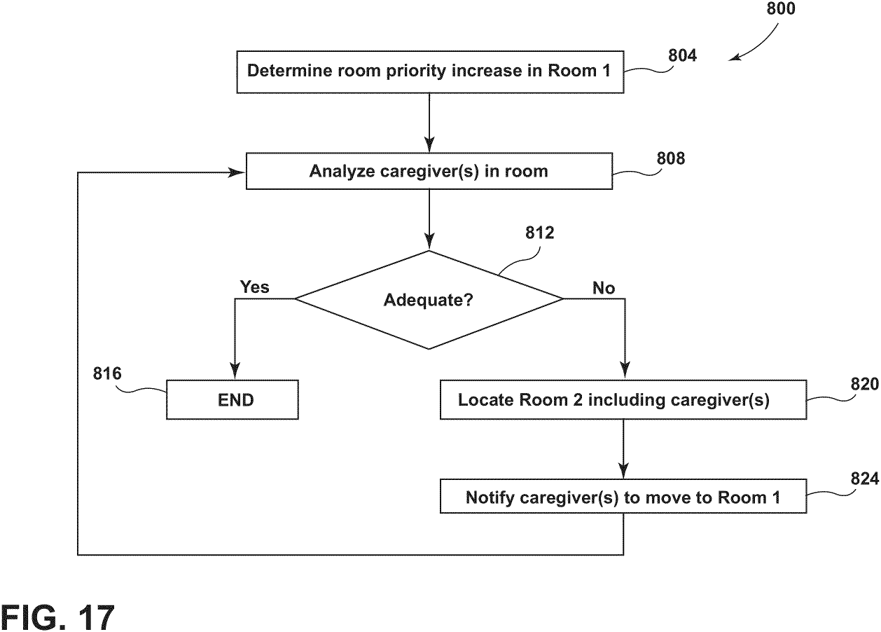

FIG. 17 is a flow diagram of a method 800 of operating a healthcare communication system. The method 800 may begin at step 804 where the healthcare communication system 370 makes a determination that a first room experiences a change in priority, such as an increase in the same. For example, the first room may have initially had a low priority status where the presence of caregivers was not necessary. Upon healthcare communication system 370 detecting an emergent situation or a non-emergent request, etc., a priority status of the first room may be increased to medium priority or high priority, requiring the presence of additional, or specifically trained caregivers. Next, at step 808, the healthcare communication system 370 analyzes the first room and determines the number and type of resources (e.g., staff members having the necessary skill sets, or qualifications associated with a communication device 10) needed to adequately attend to the reason for change in priority. A decision is made at step 812 by a processor 44 of the healthcare communication system 370 as to whether the number and type of resources in the first room is adequate. In the case that the decision is yes, and the number and type of caregivers present in the first room is adequate, the method 800 is complete at step 816. However, in the case that the decision is no, and the number and/or type caregivers present in the first room is not adequate, the method 800 continues to step 820. For example, the health care communication system 370 can determine that the first room includes a caregiver who is a licensed practical nurse. However, the health care communication system 370 received input at step 804 that the patient in the first room is experiencing cardiac arrest. Therefore, the first room now has a need for a team of providers (e.g., a code team). Accordingly, the health care communication system 370 will employ the location server 362 to find nearby caregivers having qualifications needed for the code team. At step 820, the healthcare communication system 370 locates the caregiver(s) with the required training or skills associated with communication devices 10 that are needed for the first room. In some aspects, the second room may include a greater number of caregivers associated with communication devices 10 than is needed in that room/region at that time. Next, at step 824, the healthcare communication system 370 communicates with selected communication device(s) 10 and sends an alert and request to those device(s) 10 to move to the first room. Following step 824, the method continues and returns to step 808 where the number of caregivers in the first room is analyzed again. The method 800 can continue until the appropriate, or required, number of caregivers are located in the first room.

The communication device 10 is illustrated and described for use within a healthcare facility, but may be used in other settings and/or environments. The care facility may include one or more communication devices 10 at any given time. Use of the present device may provide for a variety of advantages. The wearable communication device 10 allows for voice communication (e.g., voice assistance technology) in situations where use of hands is not practical. This can include situations that require the use of PPE equipment or in locations such as an operation theater. The communication device 10 is in the form of a small wearable device that allows voice and duress calls to be placed. The communication device 10 may include the locating beacon 50 and form a RTLS in order to more quickly locate caregivers in duress.

According to one aspect of the present disclosure, a communication device for use in a care facility comprises a housing that is configured to be worn on a caregiver. A display is disposed on the housing. A microphone is configured to detect sound signals. A speaker is configured to convert an electromagnetic wave input into a sound wave output. A controller is configured to control or receive input from the display, the microphone, the speaker, and a voice command button, where the controller is configured to authenticate the caregiver based on the detected sound signals as an authorized user having a caregiver identification unique to the caregiver.

According to another aspect of the present disclosure, a caregiver’s identification of a communication device is associated with a caregiver’s badge identification. The communication device is configured to display a code providing access to a barrier based on the caregiver’s badge identification.

According to yet another aspect of the present disclosure, an authentication of a communication device identifies an authorization level of a caregiver.

According to still another aspect of the present disclosure, an authentication of a communication device identifies a caregiver group associated with a voice command.

According to another aspect of the present disclosure, a communication device comprises a beacon that is configured to emit locating signals, where a controller is configured to receive the locating signals from the beacon and transmit the locating signals over a communication interface.

According to yet another aspect of the present disclosure, a communication device is configured to send a voice message to a second communication device and the second communication device is assigned to a compatible caregiver.

According to still another aspect of the present disclosure, a compatible caregiver includes a specific certification.

According to one aspect of the present disclosure, a healthcare communication system comprises a plurality of wearable communication devices. Each wearable communication device comprises a housing that is configured to be worn by a caregiver, a display disposed on the housing, a microphone that is configured to detect sound signals, a speaker that is configured to convert an electromagnetic wave input into a sound wave output, a beacon that is configured to emit locating signals, and a controller that is configured to control or receive signals from the display, the microphone, the speaker, and the beacon. A real-time locating system is in communication with the plurality of wearable communication devices, where the controllers of each communication device are communicatively coupled with one another to establish a communication interface between each communication device. Based upon a first location of a first wearable communication device, a voice message is sent to a second communication device. The second communication device includes a second location. The second location is within a predetermined proximity of the first location.

According to another aspect of the present disclosure, a first communication device authorizes a voice command based on a detected user’s voice and the first communication device sends a voice message to a second communication device. The second communication device is assigned to a compatible caregiver.

According to another aspect of the present disclosure, a compatible caregiver of a communication device includes a specific certification associated with an identity of a user.

According to yet another aspect of the present disclosure, authorizing a voice command of a communication system identifies a caregiver group.

According to still another aspect of the present disclosure, a first communication device of a communication system is configured to perform voice authentication, where the voice authentication identifies a unique identity of the caregiver.

According to another aspect of the present disclosure, a first communication device is configured to perform voice authentication, where the voice authentication identifies an authorization level of a caregiver.

According to yet another aspect of the present disclosure, a controller of a communication system is configured to detect a relative location of a plurality of wearable communication devices throughout a facility.

According to one aspect of the present disclosure, a method of communicating between communication devices over a healthcare communication system comprises receiving a voice command from a communication device, authorizing the voice command, identifying an action in response to the voice command, identifying a compatible communication device within a predetermined proximity, and communicating the action with the compatible communication device.

According to another aspect of the present disclosure, a method of identifying a compatible communication device within a predetermined proximity further comprises identifying all compatible communication devices within the predetermined proximity and, communicating an action, further comprises communicating the action with each of the compatible communication devices.

According to yet another aspect of the present disclosure, a method further comprises sending an acknowledgement to each compatible communication device, where the acknowledgement includes an alert that an action has been engaged by one of a plurality of compatible communication devices.

According to still another aspect of the present disclosure, a compatible communication device is assigned to a caregiver having a specific certification.

According to another aspect of the present disclosure, a compatible communication device is assigned to a caregiver authorized to deliver treatment equipment and an action requests a treatment equipment.

According to yet another aspect of the present disclosure, a compatible communication device is assigned to a robotic or automated machine authorized to deliver treatment equipment and an action requests a treatment equipment.

According to still another aspect of the present disclosure, a compatible communication device is assigned to a caregiver authorized to deliver patient supplies and an action requests a patient supply.

According to another aspect of the present disclosure, a compatible communication device is assigned to a robotic or automated machine authorized to deliver patient supplies and an action requests a patient supply.

According to yet another aspect of the present disclosure, a patient supply is at least one of a medicine, a blanket, a food, and a wound dressing.

According to still another aspect of the present disclosure, a compatible communication device is assigned to a security personnel.

According to another aspect of the present disclosure, a compatible communication device is assigned to a caregiver registered to a selected provider group of a plurality of provider groups.

According to one aspect of the present disclosure, a method of communicating between communication devices over a healthcare communication system comprises enabling an active listening mode, identifying of an action event by a first communication device, determining a response to the action event, communicating the response to a second communication device, and communicating a notification to the first communication device regarding an acknowledgement to the response to the second communication device.

According to another aspect of the present disclosure, a method where an action event comprises a code alert.

According to one aspect of the present disclosure, a method of operating a communication device comprises enabling an active listening mode, detecting at least one piece of equipment operating in a predetermined proximity, determining a conflict, determining a response to the conflict, and outputting a voice message to the communication device regarding the response.

According to another aspect of the present disclosure, a method where the at least one equipment comprises a blood oxygen warning alert and the conflict includes a delivery of medicine, further where a response includes a warning to not deliver medicine because the blood oxygen warning alert is on.

According to one aspect of the present disclosure, a method of communicating between communication devices over a healthcare communication system comprises receiving an inertial measurement from a wearable communication device, determining a recognized gesture from the inertial measurement, identifying an action in response to the recognized gesture, identifying a compatible communication device within a predetermined proximity, and communicating the action with the compatible communication device.

According to one aspect of the present disclosure, a communication device for use in a care facility comprises a housing that is configured to be worn on a caregiver, a display that is disposed on the housing, a microphone that is configured to detect sound signals, a speaker that is configured to convert an electromagnetic wave input into a sound wave output, and a controller that is configured to control or receive input from the display, the microphone, the speaker, and a voice command button, where the controller is configured to authenticate the caregiver based on the detected sound signals as an authorized user having a caregiver identification unique to the caregiver.

According to another aspect of the present disclosure, a caregiver’s identification of a communication device is associated with a caregiver badge identification and a communication device is configured to display a code providing access to a barrier based on the caregiver badge identification.

According to yet another aspect of the present disclosure, an authentication of a communication device identifies an authorization level of a caregiver.

According to still another aspect of the present disclosure, an authentication of a communication device identifies a caregiver group associated with a voice command.

According to another aspect of the present disclosure, a controller determines a direction that a detected sound signal is originating from and authenticates a caregiver based on a direction as an authorized user wearing a communication device.

According to yet another aspect of the present disclosure, a communication device is configured to send a voice message to a second communication device and the second communication device is assigned to a compatible caregiver.

According to still another aspect of the present disclosure, a compatible caregiver includes a specific certification.

According to one aspect of the present disclosure, a healthcare communication system comprises a plurality of wearable communication devices. Each wearable communication device comprises a housing configured to be worn by a caregiver, a display disposed on the housing, a microphone configured to detect sound signals, a speaker configured to convert an electromagnetic wave input into a sound wave output, a beacon configured to emit locating signals, and a controller configured to control or receive signals from the display, the microphone, the speaker, and the beacon. A real-time locating system in communication with the plurality of wearable communication devices, where the controllers of each communication device are communicatively coupled with one another to establish a communication interface between each communication device and, based upon a first location of a first wearable communication device, a voice message is sent to a second communication device, the second communication device including a second location, the second location within a predetermined proximity of the first location.

According to another aspect of the present disclosure, a first wearable communication device authorizes a voice command based on a detected user’s voice and the first wearable communication device sends a voice message to a second communication device. The second communication device is assigned to a compatible caregiver.

According to another aspect of the present disclosure, a compatible caregiver includes a specific certification associated with an identity of a user.

According to yet another aspect of the present disclosure, the first wearable communication device is configured to detect a voice command that identifies a caregiver group.

According to still another aspect of the present disclosure, a first wearable communication device is configured to perform voice authentication, where the voice authentication identifies a unique identity of the caregiver.

According to another aspect of the present disclosure, a unique identity of a caregiver is based at least in part on a voice pitch of the caregiver.

According to yet another aspect of the present disclosure, a controller identifies an action event by the first wearable communication device by receiving an inertial measurement from the first wearable communication device and the controller determines a recognized gesture from the inertial measurement, further where the recognized gesture corresponds to an emergency and a voice message sent to the second communication device comprises a request for assistance.

According to another aspect of the present disclosure, a first wearable communication device is configured to perform voice authentication, where the voice authentication identifies an authorization level of a caregiver.

According to yet another aspect of the present disclosure, a controller is configured to detect a relative location of the plurality of wearable communication devices throughout a facility and make a determination on whether a number of caregivers present in a region is appropriate.

According to one aspect of the present disclosure, a method of communicating between communication devices over a healthcare communication system comprises receiving a voice command from an origin communication device, authorizing the voice command based at least in part on a distinct noise characteristic, identifying an action in response to the voice command, identifying a compatible communication device within a predetermined proximity, and communicating the action with the compatible communication device.

According to another aspect of the present disclosure, a method where identifying a compatible communication device within a predetermined proximity further comprises identifying all compatible communication devices within the predetermined proximity and communicating an action further comprises communicating the action with each of the compatible communication devices.

According to yet another aspect of the present disclosure, a method where an origin communication device utilizes short- range communication to reduce a number of communication devices located within a predetermined proximity to only compatible communication devices that are the closest to the origin device.

According to still another aspect of the present disclosure, a method where a compatible communication device acknowledges an action and further where the compatible communication device generates an alert that an associated caregiver is moving in a conflicting direction with respect to a location of an origin device.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A communication device for use in a care facility, comprising:
a housing configured to be worn on a caregiver;
a display disposed on the housing;
a microphone configured to detect sound signals;
a speaker configured to convert an electromagnetic wave input into a sound wave output; and
a controller configured to control or receive input from the display, the microphone, the speaker, and a voice command button, wherein the controller is configured to authenticate the caregiver based on the detected sound signals as an authorized user having a caregiver identification unique to the caregiver.

2. The communication device of claim 1, wherein the caregiver identification is associated with a caregiver badge identification and the communication device is configured to display a code providing access to a barrier based on the caregiver badge identification.

3. The communication device of claim 1, wherein the authentication of the caregiver identifies an authorization level of the caregiver.

4. The communication device of claim 1, wherein the authentication identifies a caregiver group associated with a voice command.

5. The communication device of claim 1, wherein the controller determines a direction that the detected sound signals are originating from and authenticates the caregiver based on the direction as an authorized user wearing the communication device.

6. The communication device of claim 1, wherein the communication device is configured to send a voice message to a second communication device and the second communication device is assigned to a compatible caregiver.

7. The communication device of claim 6, wherein the compatible caregiver includes a specific certification.

8. A healthcare communication system, comprising:
a plurality of wearable communication devices, each wearable communication device comprising:
a housing configured to be worn by a caregiver;
a display disposed on the housing;
a microphone configured to detect sound signals;
a speaker configured to convert an electromagnetic wave input into a sound wave output;
a beacon configured to emit locating signals; and
a controller configured to control or receive signals from the display, the microphone, the speaker, and the beacon; and
a real-time locating system in communication with the plurality of wearable communication devices, wherein the controllers of each communication device are communicatively coupled with one another to establish a communication interface between each communication device and, based upon a first location of a first wearable communication device, a voice message is sent to a second communication device, the second communication device including a second location, the second location within a predetermined proximity of the first location.

9. The communication system of claim 8, wherein the first wearable communication device authorizes a voice command based on a detected user’s voice and the first wearable communication device sends the voice message to the second communication device, the second communication device assigned to a compatible caregiver.

10. The communication device of claim 9, wherein the compatible caregiver includes a specific certification associated with an identity of a user.

11. The communication system of claim 8, wherein the first wearable communication device is configured to detect a voice command that identifies a caregiver group.

12. The communication system of claim 8, wherein the first wearable communication device is configured to perform voice authentication, wherein the voice authentication identifies a unique identity of the caregiver.

13. The communication system of claim 12, wherein the unique identity of the caregiver is based at least in part on a voice pitch of the caregiver.

14. The communication system of claim 8, wherein the controller identifies an action event by the first wearable communication device by receiving an inertial measurement from the first wearable communication device and the controller determines a recognized gesture from the inertial measurement, further wherein the recognized gesture corresponds to an emergency and the voice message sent to the second communication device comprises a request for assistance.

15. The communication system of claim 8, wherein the first wearable communication device is configured to perform voice authentication, wherein the voice authentication identifies an authorization level of the caregiver.

16. The communication system of claim 8, wherein the controller is configured to detect a relative location of the plurality of wearable communication devices throughout a facility and make a determination on whether a number of caregivers present in a region is appropriate.

17. A method of communicating between communication devices over a healthcare communication system, comprising:
receiving a voice command from an origin communication device;
authorizing the voice command based at least in part on a distinct noise characteristic;
identifying an action in response to the voice command;
identifying a compatible communication device within a predetermined proximity; and
communicating the action with the compatible communication device.

18. The method of claim 17, wherein identifying a compatible communication device within a predetermined proximity further comprises identifying all compatible communication devices within the predetermined proximity and communicating the action further comprises communicating the action with each of the compatible communication devices.

19. The method of claim 18, wherein the origin communication device utilizes short range to reduce a number of communication devices located within the predetermined proximity to only compatible communication devices that are the closest to the origin device.

20. The method of claim 17, wherein the compatible communication device acknowledges the action and further wherein that the compatible communication device generates an alert that an associated caregiver is moving in a conflicting direction with respect to a location of the origin device.
